(19) United States
(12) Patent Application Publication
(10) Pub. No.: US 2011/0268809 A1
(43) Pub. Date: Nov. 3, 2011

(54) Nicotine-Containing Pharmaceutical Compositions

(76) Inventors: Paul Andrew Brinkley, Winston-Salem, NC (US); August Joseph Borschke, Winston-Salem, NC (US)

(21) Appl. No.: 12/769,335

(22) Filed: Apr. 28, 2010

(51) Int. Cl. A61K 9/68; A24F 47/00; A61P 25/34; A61K 31/465; A61K 31/787

(52) U.S. Cl. 424/499; 514/343; 424/78.15; 424/48; 424/489; 131/270

(57) ABSTRACT

A composition intended to be employed for therapeutic purposes incorporates a source of nicotine and at least one levulinate moiety. Representative forms of nicotine include free base (e.g., as a mixture of nicotine and microcrystalline cellulose), a nicotine salt (e.g., as nicotine bitartrate) or nicotine polacrilex. The levulinate moiety can have the form of an acid (e.g., levulinic acid), a levulinate salt (e.g., sodium levulinate), or an ester of levulinic acid (e.g., methyl levulinate or ethyl levulinate). The composition can incorporate nicotine and levulinic acid in a salt form (e.g., nicotine levulinate). The composition can be composed of at least two forms of nicotine, and one of the forms of nicotine is in the form of nicotine levulinate. The composition is useful for treatment of central nervous system conditions, diseases, and disorders, and as a nicotine replacement therapy.

### FIELD OF THE INVENTION

The present invention relates to compositions that contain nicotine, and in particular, to nicotine-containing pharmaceutical compositions intended to be administered to provide a pharmacological effect, or otherwise used for therapeutic purposes.

### BACKGROUND OF THE INVENTION

Central nervous system (CNS) conditions, diseases, or disorders can be drug induced; can be attributed to genetic predisposition, infection or trauma; or can be of unknown etiology. They comprise neuropsychiatric disorders, neurological diseases and mental illnesses; and include neurodegenerative diseases, behavioral disorders, cognitive disorders and cognitive affective disorders. The clinical manifestations of several CNS conditions, diseases or disorders have been attributed to CNS dysfunction (i.e., disorders resulting from inappropriate levels of neurotransmitter release, inappropriate properties of neurotransmitter receptors, and/or inappropriate interaction between neurotransmitters and neurotransmitter receptors).

Nicotinic compounds, such as nicotine, are capable of affecting nicotinic acetylcholinergic receptors (nAChRs). Subtypes of nAChRs exist in both the CNS and the peripheral nervous system (PNS), but the distribution of subtypes is heterogeneous. For instance, certain subtypes which are predominant in vertebrate brain, others predominate at the autonomic ganglia, and others predominate at neuromuscular junction. Activation of nAChRs by nicotinic compounds results in neurotransmitter release. See, for example, Dwoskin et al., Exp. Opin. Ther. Patents, 10: 1561-1581 (2000); Schmitt et al., Annual Reports in Med. Chem., 35: 41-51 (2000); Huang et al., J. Am. Chem. Soc., 127: 14401-14414 (2006); Arneric et al., Biochem. Pharmacol., 74: 1092-1101 (2007) and Millar, Biochem. Pharmacol., 78: 766-776 (2009), which are incorporated herein by reference.

It has been suggested that administration of nicotine, and other nicotinic compounds, can result in various pharmacological effects. See, for example, U.S. Pat. No. 5,583,140 to Bencherif et al.; U.S. Pat. No. 5,723,477 to McDonald et al.; U.S. Pat. No. 7,001,900 to Jacobsen et al.; U.S. Pat. No. 7,135,484 to Dart et al. and U.S. Pat. No. 7,214,686 to Bencherif et al.; and US Pat. Pub. No. 2010/0004451 to Ahmad et al., which are incorporated herein by reference. As a result, it has been suggested that nicotine, and other nicotinic compounds, can exhibit utility in the treatment of a wide variety of conditions, diseases, and disorders, including those that affect the CNS. Additionally, administration of nicotine and nicotinic compounds has been proposed for treatment of certain other conditions, diseases, and disorders. See, for example, U.S. Pat. No. 5,604,231 to Smith et al.; U.S. Pat. No. 5,811,442 to Bencherif et al.; U.S. Pat. No. 6,238,689 to Rhodes et al. and U.S. Pat. No. 6,489,349 to Bencherif et al., which are incorporated herein by reference. Furthermore, administration of nicotine has been employed in an effort to help cigarette smokers quit smoking (i.e., as a smoking cessation aid). For example, nicotine has been an active ingredient of various types of so-called “nicotine replacement therapy” or “NRT” products.

It has been proposed to administer nicotine using a transdermal patch. Representative types of nicotine-containing transdermal patch products have been marketed under the tradenames “Habitrol,” “Nicoderm,” “Nicorette,” “Nicorette CQ,” “Nicotinell” and “ProStep.” See also, for example, U.S. Pat. No. 4,597,961 to Etscom; U.S. Pat. No. 5,298,257 to Bannon et al.; U.S. Pat. No. 5,603,947 to Wong et al.; U.S. Pat. No. 5,834,011 to Rose et al.; U.S. Pat. No. 6,165,497 to Osborne et al. and U.S. Pat. No. 6,676,959 to Anderson et al., which are incorporated herein by reference. It also has been suggested that transdermal administration of nicotine can be accompanied by ingestion of other types of nicotine-containing products. See, for example, U.S. Pat. No. 5,593,684 to Baker et al.; US Pat. Pub. No. 2009/0004249 to Gonda; and Fagerstrom, Health Values, 18:15 (1994), which are incorporated herein by reference.

One particularly popular way to provide for oral administration of nicotine has been through the use of nicotine-containing gum. Nicotine-containing gum products have been marketed under the tradenames “Nicorette,” “Nicotinell” and “Zonnic.” See also, for example, U.S. Pat. No. 3,845,217 to Ferno et al.; U.S. Pat. No. 3,877,468 to Lichtneckert et al.; U.S. Pat. No. 3,901,248 to Lichtneckert et al.; U.S. Pat. No. 6,344,222 to Cherukuri et al.; U.S. Pat. No. 6,358,060 to Pinney et al.; U.S. Pat. No. 6,773,716 to Ream et al. and U.S. Pat. No. 6,893,654 to Pinney et al.; and US Pat. Pub. No. 2004/0191322 to Hansson, which are incorporated herein by reference.

Another way that has been employed to provide oral administration of nicotine has been through the use of nicotine-containing lozenge or tablet types of products. Nicotine-containing lozenge, mini lozenge, tablet, and microtab types of products have been marketed under the tradenames “Commit,” “Nicorette,” “Nicotinell” and “NiQuitin.” See also, for example, U.S. Pat. No. 5,110,605 to Acharya; U.S. Pat. No. 5,733,574 to Dam; U.S. Pat. No. 6,280,761 to Santus; U.S. Pat. No. 6,676,959 to Andersson et al. and U.S. Pat. No. 6,248,760 to Wilhelmsen; US Pat. Pub. Nos. 2001/0016593 to Wilhelmsen and 2010/0004294 to Axelsson et al., which are incorporated herein by reference.

Nicotine also has been administered in the form of nasal or oral sprays. Various exemplary ways to administer nicotine in the form of a nasal spray are set forth in U.S. Pat. No. 4,579,858 to Ferno et al.; U.S. Pat. No. 5,656,255 to Jones and U.S. Pat. No. 6,596,740 to Jones, which are incorporated herein by reference. Various exemplary ways to administer nicotine in the form of an oral spray, such as for buccal administration, are set forth in U.S. Pat. No. 6,024,097 to Von Wielligh; US Pat. Pub. Nos. 2003/0159702 to Lindell et al.; 2007/0163610 to Lindell et al. and 2009/0023819 to Axelsson; EP 1458388 to Lindell et al.; and PCT WO 2008/037470 to Axelsson et al., which are incorporated herein by reference. Nicotine-containing sprays have been marketed under the tradenames “Nicotrol NS,” “Quit” and “Zonnic.”

Various other ways to administer nicotine for the purpose of providing a therapeutic effect have been proposed. For example, it has been suggested that nicotine can be incorporated into orally dissolving films (e.g., U.S. Pat. No. 6,709,671 to Zerbe et al.; U.S. Pat. No. 7,025,983 to Leung et al.; and U.S. Pat. No. 7,491,406 to Leung et al.; and US Pat. Pub. Nos. 2006/0198873 to Chan et al. and 2006/0204559 to Bess et al.); oral osmotic devices (e.g., U.S. Pat. No. 5,147,654 to Place et al.); gum pads (e.g., U.S. Pat. No. 6,319,510 to Yates); oral patches (e.g., US Pat. Pub. No. 2006/0240087 to Houze et al.); snuff-type forms in pouches or sachets (e.g., U.S. Pat. No. 4,907,605 to Ray et al. and US Pat. Pub. No. 2009/0293895 to Axelsson et al.); lip balm (e.g., U.S. Pat. No. 7,105,173 to Rolling) and beverages (e.g., U.S. Pat. No. 6,268,386 to Thompson; U.S. Pat. No. 7,115,297 to Stillman and U.S. Pat. No. 7,435,749 to Knight). It also has been suggested that nicotine can be delivered using various types of inhalation devices and vapor delivery systems (e.g., U.S. Pat. No. 4,284,809 to Ray; U.S. Pat. No. 4,800,903 to Ray et al.; U.S. Pat. No. 6,234,169 to Bulbrook et al. and U.S. Pat. No. 6,874,507 to Farr; and US Pat. Pub. Nos. 2006/0018840 to Lechuga-Ballesteros and 2009/0005423 to Gonda; and EP 1,618,803 to Hon).

It would be desirable to provide a composition capable of delivering or administering nicotine via an oral or nasal route for therapeutic purposes.

### SUMMARY OF THE INVENTION

In one aspect, the present invention relates to a nicotine-containing composition intended to be employed for therapeutic purposes. The composition is typically in a pharmaceutically acceptable form adapted for oral or nasal delivery of the composition, preferably oral delivery. The composition incorporates at least one source of nicotine and at least one levulinate moiety, and typically at least a portion of the nicotine is in the form of a salt with the levulinate moiety. A composition adapted for oral or nasal delivery can be enhanced by utilizing a levulinate moiety as an excipient, wherein the levulinate moiety is employed in an amount sufficient to reduce the negative sensory characteristics sometimes associated with oral delivery of nicotine.

The levulinate moiety can have the form of an acid, an ionic salt of levulinic acid (e.g., alkali metal or alkali earth metal salt such as calcium levulinate, magnesium levulinate, sodium levulinate, or potassium levulinate), or an ester of levulinic acid (e.g., methyl levulinate or ethyl levulinate). In one embodiment, the composition incorporates nicotine and levulinic acid in a salt form (i.e., the levulinate moiety is incorporated within nicotine levulinate).

Typically, compositions of the invention include at least one additional form of nicotinic compound in addition to nicotine levulinate. In other words, a composition of the invention that incorporates a source of nicotine active ingredient is typically composed of at least two forms of nicotine, and one of the forms of nicotine is in the form of nicotine levulinate. The other form of nicotine can be as a free base (e.g., as a mixture of nicotine free base and a porous particulate carrier such as microcrystalline cellulose), as another form of nicotine salt (e.g., as nicotine bitartrate or another organic acid salt of nicotine), as a resin complex of nicotine (e.g., nicotine polacrilex), or as a solvate, or other suitable form.

In certain embodiments, one or both of the nicotinic compound and the levulinate moiety are sorbed onto a porous particulate carrier such as microcrystalline cellulose. For example, both a nicotine free base and nicotine levulinate can be sorbed onto the porous particulate carrier.

In one embodiment, the nicotine-containing pharmaceutical composition comprises a source of nicotine selected from the group consisting of nicotine in free base form, a nicotine salt (other than nicotine levulinate), a resin complex of nicotine, and mixtures thereof, and a levulinate moiety selected from the group consisting of levulinic acid, nicotine levulinate, an alkali metal or alkali earth metal salt of levulinic acid, an alkyl ester of levulinic acid, and mixtures thereof; wherein the composition is in a pharmaceutically acceptable form adapted for oral ingestion of the composition.

Compositions of the present invention, including compositions incorporating other pharmaceutically acceptable excipient ingredients, can be provided in forms suitable for administration to human subjects, particularly in forms adapted for oral ingestion. Exemplary formats and configurations for oral administration of nicotine-containing compositions for therapeutic purposes include gum, tablet, lozenge, pouch, and mouth-spray types of products.

In another aspect, the present invention relates to a method for treating a condition, disease, or disorder responsive to stimulation of nicotinic acetylcholinergic receptors, comprising orally or nasally administering an effective amount of a pharmaceutical composition according to any of the embodiments noted herein to a human subject in need of treatment.

In one regard, the method involves administering a composition that incorporates a source of nicotine and a levulinate moiety (e.g., as an excipient). At least a portion of the nicotine within the composition typically possesses the form of a free base (e.g., as a mixture of nicotine and microcrystalline cellulose), or a nicotine salt (e.g., as nicotine bitartrate), or nicotine polacrilex.

Exemplary conditions that can be treated include disorders of the central nervous system. Additionally, the compositions of the invention can be used as a smoking cessation aid.

### DETAILED DESCRIPTION OF THE PREFERRED EMBODIMENTS

The present inventions now will be described more fully hereinafter. The invention may be embodied in many different forms and should not be construed as limited to the embodiments set forth herein; rather, these embodiments are provided so that this disclosure will satisfy applicable legal requirements. As used in this specification and the claims, the singular forms “a,” “an,” and “the” include plural referents unless the context clearly dictates otherwise.

The present invention involves the use of nicotinic compounds for therapeutic purposes and provides compositions adapted for oral or nasal delivery of nicotinic compounds. As used herein, “nicotinic compound” or “source of nicotine” refers to naturally-occurring or synthetic nicotine unbound from a plant material, meaning the compound is at least partially purified and not contained within a plant structure such as a tobacco leaf. Most preferably, nicotine is naturally-occurring and obtained as an extract from a Nicotiana species (e.g., tobacco). The nicotine can have the enantiomeric form S(−)-nicotine, R(+)-nicotine, or a mixture of S(−)-nicotine and R(+)-nicotine. Most preferably, the nicotine is in the form of S(−)-nicotine (e.g., in a form that is virtually all S(−)-nicotine) or a racemic mixture composed primarily or predominantly of S(−)-nicotine (e.g., a mixture composed of about 95 weight parts S(−)-nicotine and about 5 weight parts R(+)-nicotine). Most preferably, the nicotine is employed in virtually pure form or in an essentially pure form. Highly preferred nicotine that is employed has a purity of greater than about 95 percent, more preferably greater than about 98 percent, and most preferably greater than about 99 percent, on a weight basis. Despite the fact that nicotine can be extracted from Nicotiana species, it is highly preferred that the nicotine (and the composition and products produced in accordance with the present invention) are virtually or essentially absent of other components obtained from or derived from tobacco.

Nicotinic compounds of the invention can include nicotine in free base form, salt form, as a complex, or as a solvate. See, for example, the discussion of nicotine in free base form in US Pat. Pub. No. 2004/0191322 to Hansson, which is incorporated herein by reference. At least a portion of the nicotinic compound can be employed in the form of a resin complex of nicotine, where nicotine is bound in an ion exchange resin, such as nicotine polacrilex. See, for example, U.S. Pat. No. 3,901,248 to Lichtneckert et al., which is incorporated herein by reference. At least a portion of the nicotine can be employed in the form of a salt. Salts of nicotine can be provided using the types of ingredients and techniques set forth in U.S. Pat. No. 2,033,909 to Cox et al. and Perfetti, Beitrage Tabakforschung Int., 12: 43-54 (1983), which are incorporated herein by reference. Additionally, salts of nicotine have been available from sources such as Pfaltz and Bauer, Inc. and K&K Laboratories, Division of ICN Biochemicals, Inc.

Exemplary pharmaceutically acceptable nicotine salts include nicotine salts of tartrate (e.g., nicotine tartrate and nicotine bitartrate) chloride (e.g., nicotine hydrochloride and nicotine dihydrochloride), sulfate, perchlorate, ascorbate, fumarate, citrate, malate, lactate, aspartate, salicylate, tosylate, succinate, pyruvate, and the like; nicotine salt hydrates (e.g., nicotine zinc chloride monohydrate), and the like. Additional organic acids that can form salts with nicotine include formic, acetic, propionic, isobutyric, butyric, alpha-methylbutyric, isovaleric, beta-methylvaleric, caproic, 2-furoic, phenylacetic, heptanoic, octanoic, nonanoic, oxalic, malonic, and glycolic acid, as well as other fatty acids having carbon chains of up to about 20 carbon atoms.

The compositions of the invention also include a levulinate moiety. As used herein, “levulinate moiety” refers to levulinic acid or an ionic salt or ester of levulinic acid. Accordingly, a levulinic moiety used in the invention can be provided in a variety of forms, including free acid form, or in the form of an ionic salt or an ester, or as a mixture of a variety of forms (e.g., mixture of free acid and sodium salt). Exemplary salt forms include alkali metal and alkali earth metal salts (e.g., calcium levulinate, magnesium levulinate, sodium levulinate, and potassium levulinate). Exemplary esters include alkyl esters of levulinic acid (e.g., methyl levulinate or ethyl levulinate). See also, for example, U.S. Pat. No. 4,830,028 to Lawson et al. and U.S. Pat. No. 5,031,646 to Lippiello et al.; and Leonard, Ind. Eng. Chem., 48: 1331-1341 (1956), which are incorporated herein by reference.

In one embodiment, the levulinate moiety can be employed in the form of a salt component formed in conjunction with the nicotinic compound active ingredient (e.g., as a component of a nicotine levulinate salt). The levulinate moiety also can be incorporated within the composition in at least two forms (e.g., as a sodium levulinate salt in combination with levulinic acid).

Incorporating a levulinate moiety into a nicotine-containing pharmaceutical composition intended for oral or nasal delivery can ameliorate the types of dissonant sensory and organoleptic effects attributed to the administration of nicotine. In essence, the levulinate moiety acts as a carrier or excipient for nicotine in a manner that reduces the harsh sensory characteristics sometimes associated with oral or nasal delivery of nicotine.

In many embodiments, the nicotinic compound will be present in multiple forms, wherein at least one of the forms is typically a salt with the levulinate moiety (e.g., nicotine levulinate). For example, the nicotine can be employed within the composition as a mixture of at least two salts (e.g., two different organic acid salts including nicotine levulinate), as at least two salts that are segregated within the composition, in a free base form and salt form, in a free base form and a salt form that are segregated within the composition, in a salt form and in a complexed form (e.g., a resin complex such as nicotine polacrilex), in a salt for and in a complexed form that are segregated with in the composition, in a free base form and a complexed form, in a free base form and a complexed form that are segregated within the composition, or the like. As such, each single dosage unit or piece (e.g., gum piece, lozenge, sachet, etc.) can incorporate at least two forms of nicotine.

The compositions of the invention possess a form that is pharmaceutically effective and pharmaceutically acceptable. That is, the composition most preferably does not incorporate to any appreciable degree, or does not purposefully incorporate, components of tobacco, other than nicotine. As such, pharmaceutically effective and pharmaceutically acceptable compositions do not include tobacco, processed tobacco components, or many of the components of tobacco traditionally present within tobacco-containing cigarettes, cigars, pipes, or smokeless forms of tobacco products. Highly preferred compositions that are derived by extracting naturally-occurring nicotine from tobacco include less than 0.5 weight percent of tobacco components other than nicotine, more often less than about 0.25 weight percent, and typically are entirely absent or devoid of components of tobacco, processed tobacco components, or components derived from tobacco, other than nicotine, based on the total weight of the composition.

The pharmaceutical compositions of the invention may be conveniently made available in a unit dosage form, whereby such formulations may be prepared by any of the methods generally known in the pharmaceutical arts. Such methods of preparation comprise combining (by various methods) an active agent with a suitable carrier or other adjuvant, which may consist of one or more ingredients. The combination of the active ingredient with the one or more adjuvants is then physically treated to present the formulation in a suitable form for delivery (e.g., shaping into a tablet or forming an aqueous suspension).

The nicotine-containing pharmaceutical compositions of the invention can incorporate various pharmaceutically acceptable excipients in addition to the levulinate moiety. By “pharmaceutically acceptable carrier” or “pharmaceutically acceptable excipient” is intended a carrier or excipient that is conventionally used in the art to facilitate the storage, administration, and/or the healing effect of an active agent (e.g., a nicotinic compound). The carrier(s) are preferably pharmaceutically acceptable in the sense of being compatible with the other ingredients of the formulation and not unduly deleterious to the recipient thereof. A carrier may also reduce any undesirable side effects of the agent. See, Wang et al., J. Parent. Drug Assn., 34(6): 452-462 (1980), which is incorporated herein by reference. Exemplary pharmaceutical excipients and/or additives suitable for use in the compositions according to the invention are listed in Remington: The Science & Practice of Pharmacy, 21^{st} ed., Lippincott Williams & Wilkins (2006); in the Physician's Desk Reference, 64^{th} ed., Thomson PDR (2010); and in Handbook of Pharmaceutical Excipients, 6^{th} ed., Eds. Raymond C. Rowe et al., Pharmaceutical Press (2009), which are incorporated herein by reference.

The various excipients can vary, and the selection and amount of each excipient can depend upon factors such as the ultimate form and function of product that is desired. See, for example, the types of ingredients, relative amounts and combinations of ingredients, nicotine-containing formulations and preparation processes for nicotine-containing products set forth in U.S. Pat. No. 5,512,306 to Carlsson et al.; U.S. Pat. No. 5,525,351 to Dam; U.S. Pat. No. 5,549,906 to Santus; U.S. Pat. No. 5,711,961 to Reiner et al.; U.S. Pat. No. 5,811,126 to Krishnamurthy; U.S. Pat. No. 5,939,100 to Albrechtsen et al.; U.S. Pat. No. 6,024,981 to Khankari et al.; U.S. Pat. No. 6,083,531 to Humbert-Droz et al.; U.S. Pat. No. 6,090,401 to Gowan, Jr. et al.; U.S. Pat. No. 6,110,495 to Dam; U.S. Pat. No. 6,248,760 to Wilhelmsen; U.S. Pat. No. 6,280,761 to Santus; U.S. Pat. No. 6,426,090 to Ream et al.; U.S. Pat. No. 6,569,463 to Patel et al.; U.S. Pat. No. 6,583,160 to Smith et al.; U.S. Pat. No. 6,585,997 to Moro et al.; U.S. Pat. No. 6,676,959 to Andersson et al.; U.S. Pat. No. 6,893,654 to Pinney et al.; U.S. Pat. No. 7,025,983 to Leung et al. and U.S. Pat. No. 7,163,705 Johnson et al.; US Pat. Pub. Nos. 2003/0176467 to Andersson et al.; 2003/0235617 to Martino et al.; 2004/0096501 to Vaya et al.; 2004/0101543 to Liu et al.; 2004/0191322 to Hansson; 2005/0053665 to Ek et al.; 2005/0123502 to Chan et al.; 2008/0038209 to Andersen et al.; 2008/0286341 to Andersson et al.; 2009/0023819 to Axelsson; 2009/0092573 to Andersen; 2010/0004294 to Axelsson et al. and 2010/0061940 to Axelsson et al., which are incorporated herein by reference.

Representative types of excipients that are particularly useful for the manufacture of nicotine-containing products include fillers or carriers for active ingredients (e.g., calcium polycarbophil, microcrystalline cellulose, cornstarch, silicon dioxide or calcium carbonate), thickeners, film formers and binders (e.g., hydroxypropyl cellulose, hydroxypropyl methylcellulose, acacia, sodium alginate, xanthan gum and gelatin), buffers and pH control agents (e.g., magnesium oxide, magnesium hydroxide, potassium carbonate, sodium carbonate, potassium bicarbonate, sodium bicarbonate, or mixtures thereof), antiadherents (e.g., talc), glidants (e.g., colloidal silica), natural or artificial sweeteners (e.g., saccharin, acesulfame K, aspartame, sucralose, isomalt, lactose, mannitol, sorbitol, xylitol and sucrose), humectants (e.g., glycerin), preservatives and antioxidants (e.g., sodium benzoate and ascorbyl palmitate), surfactants (e.g., polysorbate 80), natural or artificial flavors (e.g., mint, cinnamon, cherry or other fruit flavors), dyes or pigments (e.g., titanium dioxide or D&C Yellow No. 10), and lubricants or processing aids (e.g, calcium stearate or magnesium stearate). Certain types of nicotine-containing products also can have outer coatings composed of ingredients capable of providing acceptable outer coatings (e.g., an outer coating can be composed of ingredients such as carnauba wax, and pharmaceutically acceptable forms of shellacs, glazing compositions and surface polish agents).

Representative compositions incorporating nicotine as an active ingredient can have various types of formats and configurations, and as a result, the character, nature, behavior, consistency, shape, form, size and weight of the composition can vary. The shape of a representative composition can be generally spherical, cylindrical (e.g., ranging form the general shape of a flattened disc to the general shape of a relatively long, slender stick), helical, obloid, square, rectangular, or the like; or the composition can have the form of a bead, granular powder, crystalline powder, capsule, film, strip, gel, or the like. The shape of the composition can resemble a wide variety of pill, tablet, lozenge, mini lozenge, capsule, caplet, pouch and gum types of products that traditionally have been employed for the administration of pharmaceutical types of products. The general nature of a representative composition can be soft or hard to the touch, or of intermediate softness or hardness; and as such, the composition can be considered to be malleable, flexible, chewy, resilient, brittle, or the like. When administered orally, various components of the product can be considered to be readily dispersible or slow to disperse, or those various components can dissolve at varying rates (e.g., from relatively fast to relatively slow). As a result, for compositions ingested by insertion in the mouth of the human subject, the release rate of active ingredient during use of the product can vary from relatively fast to relatively slow, depending upon factors such as the design of the product and the use of product by the subject using that product. See also, by way of example, the types of products proposed in U.S. Pat. No. 4,655,231 to Ray et al.; U.S. Pat. No. 5,147,654 to Place et al.; U.S. Pat. No. 5,543,424 to Carlsson et al.; U.S. Pat. No. 6,268,386 to Thompson; U.S. Pat. No. 6,319,510 to Yates; U.S. Pat. No. 6,488,953 Halliday et al.; U.S. Pat. No. 6,709,671 to Zerbe et al.; U.S. Pat. No. 7,025,983 to Leung et al.; U.S. Pat. No. 7,105,173 to Rolling; U.S. Pat. No. 7,115,297 to Stillman; U.S. Pat. No. 7,435,749 to Knight and U.S. Pat. No. 7,491,406 to Leung et al.; and US Pat. Pub. Nos. 2004/0191322 to Hansson; 2006/0198873 to Chan et al.; 2006/0240087 to Houze et al.; 2006/0204559 to Bess et al.; 2007/0269492 to Steen et al.; 2008/0020050 to Chau et al.; 2008/0286340 to Andersson et al.; 2008/0292683 to Sanghvi et al. and 2009/0004248 to Bunick et al., which are incorporated herein by reference.

Formulations of the present invention may include short-term, rapid-onset, rapid-offset, controlled release, sustained release, delayed release, and pulsatile release formulations, providing the formulations achieve administration of a nicotinic compound as described herein. See Remington's Pharmaceutical Sciences, 18^{th} ed.; Mack Publishing Company, Eaton, Pa., (1990), which is incorporated herein by reference.

Solid dosage forms may be formulated so as to provide a delayed release of the active agent (i.e., the nicotinic compound), such as by application of a coating. Delayed release coatings are known in the art, and dosage forms containing such may be prepared by any known suitable method. Such methods generally involve application of a delayed release coating composition after preparation of the solid dosage form (e.g., a tablet or caplet). Application of the coating can be by methods such as airless spraying, fluidized bed coating, use of a coating pan, or the like. Materials for use as a delayed release coating can be polymeric in nature, such as cellulosic material (e.g., cellulose butyrate phthalate, hydroxypropyl methylcellulose phthalate, and carboxymethyl ethylcellulose), and polymers and copolymers of acrylic acid, methacrylic acid, and esters thereof.

Solid dosage forms according to the present invention may also be sustained release (i.e., releasing the active agent over a prolonged period of time), and may or may not also be delayed release. Sustained release formulations are known in the art and are generally prepared by dispersing the active ingredient within a matrix of a gradually degradable or hydrolyzable material, such as an insoluble plastic, a hydrophilic polymer, or a fatty compound. Alternatively, a solid dosage form may be coated with such a material.

The manners and methods used to formulate and manufacture the composition can vary. Typical conditions associated with manufacture of pharmaceutical types of products include control of heat and temperature (i.e., the degree of heat to which the various ingredients are exposed during manufacture and the temperature of the manufacturing environment), moisture content (e.g., the degree of moisture present within individual ingredients and within the final composition), humidity within the manufacturing environment, atmospheric control (e.g., nitrogen atmosphere), airflow experienced by the various ingredients during the manufacturing process, and other similar types of factors. Additionally, various process steps involved in product manufacture can involve selection of certain solvents and processing aids, use of heat and radiation, refrigeration and cryogenic conditions, ingredient mixing rates, and the like. The manufacturing conditions also can be controlled due to selection of the form of various ingredients (e.g., solid, liquid, or gas), particle size or crystalline nature of ingredients of solid form, concentration of ingredients in liquid form, or the like. Ingredients can be processed into the desired composition by techniques such as extrusion, compression, spraying, and the like.

The manners and methods for incorporating the levulinate moiety into the nicotine-containing composition can vary. The location of the levulinate moiety within the composition can also vary. The levulinate moiety can be located throughout the composition or in selected regions of the composition (e.g., homogeneously throughout the composition, in an outer coating of the composition or in the region of the composition occupied by nicotine or in selected layer(s) of a laminated composition). As such, certain regions of the composition can be essentially devoid of the levulinate moiety, or there can exist a concentration gradient of levulinate moiety within or throughout the composition, or a certain region of the composition can have a relatively high concentration of levulinate moiety relative to other regions of that composition. Compositions can be co-extruded, laminated or formed so as to have sandwich-type forms; and hence the location of nicotine, levulinate moiety and other ingredients can be controlled in order to provide the desired features such as performance, behavior, interaction or non-interaction with other ingredients, storage stability, and the like. In addition, mixtures of component ingredients can be formulated and manufactured into core/shell types of configurations (e.g., gum or lozenge types of products that have an inner region and at least one additional overlayer), with the various regions of such products having differing overall compositions or properties. Thus, for example, the levulinate moiety can have a relatively high concentration towards the inner region of the product, or a relatively high concentration towards the outer region of the product.

Nicotine levulinate can be mixed with other forms of nicotine (e.g., with other nicotine salts or nicotine free base or nicotine polacrilex), and incorporated into the composition as a mixture. Nicotine levulinate and other forms of nicotine also can be introduced into the composition at different times or stages of the manufacturing process, or in combination with different ingredients employed in the manufacturing process. Alternatively, nicotine levulinate can be segregated from other forms of nicotine within the composition (e.g., by physically locating the various forms of nicotine at separate locations within the composition, or by segregating the forms of nicotine using encapsulation or other types of chemical means to separate those components).

In one embodiment, one or both of the nicotinic compound and the levulinate moiety are sorbed onto a porous particulate carrier material, such as microcrystalline cellulose (MCC). In one embodiment, the MCC materials used in the invention have an average particle size range of about 15 to about 250 microns. Exemplary MCC materials include various grades of AVICEL® and VIVACEL® materials. See, for example, US Pat. Pub. No. 2004/0191322 to Hansson, which is incorporated herein by reference. Thus, in certain embodiments, multiple forms of nicotinic compounds could be sorbed onto the particulate carrier including any of the various nicotinic compound combinations discussed herein, such as nicotine free base combined with nicotine levulinate, two nicotine salts of organic acids (e.g., a nicotine levulinate/nicotine tartrate mixture or a nicotine levulinate/nicotine bitartrate mixture), and the like. The nicotine compound and the levulinate moiety can be sorbed onto the particulate carrier by, for example, dissolving the levulinate moiety and the nicotinic compound in a hydrophilic solvent (e.g., water, alcohol, or mixtures thereof) and combining the solution with the particulate carrier, followed by drying to remove the solvent. The particulate carrier material with the sorbed nicotine and levulinate moiety can be combined with other carriers or excipients in order to provide a composition adapted for oral or nasal delivery of the active ingredient.

One particularly preferred type of a representative composition incorporating nicotine as an active ingredient, and that provides nicotine in a non-inhalable form, has the form of a gum or other type of similarly chewable product. Gum forms of product include gum base (e.g., typically the types of pharmaceutically acceptable gum bases available from sources such as Gum Base Co. S.p.a., Wm. J. Wrigley Jr. Company or Gumlink A/S). See, for example, the types of nicotine-containing gums, gum formulations, gum formats and configurations, gum characteristics and techniques for formulating or manufacturing gums set forth in U.S. Pat. No. 3,845,217 to Ferno et al.; U.S. Pat. No. 3,877,468 to Lichtneckert et al.; U.S. Pat. No. 3,901,248 to Lichtneckert et al.; U.S. Pat. No. 5,154,927 to Song et al.; U.S. Pat. No. 6,322,806 to Ream et al.; U.S. Pat. No. 6,344,222 to Cherukuri et al.; U.S. Pat. No. 6,355,265 to Ream et al.; U.S. Pat. No. 6,358,060 to Pinney et al.; U.S. Pat. No. 6,773,716 to Ream et al.; U.S. Pat. No. 6,893,654 to Pinney et al.; U.S. Pat. No. 7,101,579 Athanikar et al.; U.S. Pat. No. 7,163,705 to Johnson et al. and U.S. Pat. No. 7,208,186 to Norman et al.; US Pat. Pub. Nos. 2004/0194793 to Lindell et al.; 2006/0099300 to Andersen et al.; 2006/0121156 to Andersen et al.; 2006/0165842 to Andersen et al.; 2006/0204451 to Salini; 2006/0246174 to Andersen et al.; 2006/0275344 to Mody et al.; 2007/0014887 to Cherukuri et al.; 2007/0269386 to Steen et al.; 2009/0092573 to Andersen and 2010/0061940 to Axelsson et al.; which are incorporated herein by reference. The amount of composition contained within each piece or unit of gum type of product can vary. For example, a representative unit for gum products generally weighs at least about 0.5 g, often at least about 1 g, and frequently at least about 1.5 g; while the weight of a representative unit for such products generally does not exceed about 3 g, often does not exceed about 2.5 g, and frequently does not exceed about 2 g. The time period over which a gum piece can be chewed can vary; and typically, each piece of gum is chewed for at least about 5 minutes, often at least about 10 minutes, while each piece of gum typically is chewed for up to about 40 minutes, often up to about 30 minutes.

Another particularly preferred type of a representative composition incorporating nicotine as an active ingredient, and that provides nicotine in a non-inhalable form, has the form of a lozenge, mini lozenge, tablet, microtab, or other tablet-type product. See, for example, the types of nicotine-containing lozenges, lozenge formulations, lozenge formats and configurations, lozenge characteristics and techniques for formulating or manufacturing lozenges set forth in U.S. Pat. No. 4,967,773 to Shaw; U.S. Pat. No. 5,110,605 to Acharya; U.S. Pat. No. 5,733,574 to Dam; U.S. Pat. No. 6,280,761 to Santus; U.S. Pat. No. 6,676,959 to Andersson et al.; U.S. Pat. No. 6,248,760 to Wilhelmsen and U.S. Pat. No. 7,374,779 to Chen et al.; US Pat. Pub. Nos. 2001/0016593 to Wilhelmsen; 2004/0101543 to Liu et al.; 2006/0120974 to Mcneight; 2008/0020050 to Chau et al.; 2009/0081291 to Gin et al. and 2010/0004294 to Axelsson et al.; and PCT WO 91/09599 to Carlsson et al., which are incorporated herein by reference. The amount of the composition of the invention contained within each piece or unit of lozenge type of product can vary. For example, a representative unit for lozenge products generally weighs at least about 100 mg, often at least about 200 mg, and frequently at least about 300 mg; while the weight of a representative unit for such products generally does not exceed about 1.5 g, often does not exceed about 1 g, and frequently does not exceed about 0.75 g.

Another particularly preferred type of a representative composition incorporating nicotine as an active ingredient, and that provides nicotine in a non-inhalable form, has the form of a pouch or sachet type of product. See, for example, the types of pouch materials and nicotine-containing formulations set forth in US Pat. Pub. No. 2009/0293895 to Axelsson et al., which is incorporated herein by reference. See also, for example, the types of pouch materials and pouch manufacturing techniques (e.g., pouch filling and sealing techniques) set forth in US Pat. Pub. No. 2010/0018539 to Brinkley et al., which is incorporated herein by reference. The amount of composition contained within each pouch can vary. For example, a representative pouch product generally contains at least about 75 mg, often at least about 100 mg, and frequently at least about 150 mg, of composition according to the invention; while the amount of composition contained in a single representative pouch generally does not exceed about 500 mg, often does not exceed about 400 mg, and frequently does not exceed about 300 mg.

The amount of active ingredient within the overall composition can vary. For a composition intended for oral consumption by insertion into the mouth of the subject (e.g., chewable piece of gum product, a lozenge, a pouch product, or the like), the amount of nicotine within each dosage piece or unit typically is at least about 0.5 mg, generally is at least 1 mg, often is at least about 1.5 mg, and frequently is at least about 2 mg; while the amount of nicotine within each piece typically does not exceed about 10 mg, generally does not exceed about 8 mg, often does not exceed about 6 mg, and frequently does not exceed about 5 mg, calculated as nicotine base. Exemplary types of such products can incorporate about 2 mg, about 2.5 mg, about 3 mg, about 3.5 mg and about 4 mg of nicotine per piece or unit, calculated as nicotine base.

Another particularly preferred type of a representative composition incorporating nicotine as an active ingredient has the form of a spray. Preferably, such sprays are applied within the nose or mouth for absorption through nasal or oral mucosa, as opposed to a vapor or fine aerosol that is inhaled into the lungs. See, for example, the types of spray materials and nicotine-containing spray formulations set forth in U.S. Pat. No. 4,579,858 to Ferno et al.; U.S. Pat. No. 5,656,255 to Jones; U.S. Pat. No. 6,024,097 to Von Wielligh and U.S. Pat. No. 6,596,740 to Jones; US Pat. Pub. Nos. 2003/0159702 to Lindell et al.; 2007/0163610 to Lindell et al. and 2009/0023819 to Axelsson; EP 1458388 to Lindell et al.; and PCT WO 2008/037470 to Axelsson et al., which are incorporated herein by reference. Preferred spray products produce sprays or mists using nebulizers or other types of devices for producing aerosols by mechanical means. Preferred spray products employ liquid solvents or carriers (e.g., water or water/ethanol mixtures) that contain nicotine and the levulinate moiety. The concentration of the nicotine within the liquid spray formulation can vary, but typically is in the range of about 0.5 percent to about 5 percent, often about 1 percent to about 3 percent, based on the total weight of the liquid formulation and calculated as nicotine base.

Although the compositions of the invention are preferably non-inhalable, it is possible to formulate the above-noted combinations of a nicotinic compound and a levulinate moiety in a form capable of pulmonary delivery using various types of inhalation devices and vapor delivery systems designed to deliver an active agent to the lungs as opposed to buccal, sublingual, or nasal delivery. See, for example, the types of inhalable formulations and vapor delivery devices and systems set forth in U.S. Pat. No. 4,284,809 to Ray; U.S. Pat. No. 4,800,903 to Ray et al.; U.S. Pat. No. 5,167,242 to Turner et al.; U.S. Pat. No. 6,098,632 to Turner et al.; U.S. Pat. No. 6,234,169 to Bulbrook et al. and U.S. Pat. No. 6,874,507 to Farr; US Pat. Pub. Nos. 2004/0034068 to Warchol et al; 2006/0018840 to Lechuga-Ballesteros; 2008/0302375 to Andersson et al. and 2009/0005423 to Gonda; and EP 1,618,803 to Hon, which are incorporated herein by reference.

The compositions of the present invention generally incorporate a pharmaceutically effective amount of levulinate moiety. For compositions of the present invention, the amount of levulinate moiety present within the composition can vary. The amount of levulinate moiety (e.g., determined as levulinate anion) relative to the total amount of nicotine within the composition typically is at least about 10 percent, generally is at least about 20 percent, and often at least about 30 percent of the total amount of nicotine (calculated as nicotine base) within the composition, on a weight basis. The ratio of levulinate moiety to the total amount of nicotine (calculated as nicotine base) within the composition typically does not exceed about 2:1, generally does not exceed about 1.5:1, often does not exceed about 1:1, and frequently does not exceed about 0.8:1 of the total amount of nicotine within the composition, based on the weight of nicotine base and levulinate anion within the composition.

Certain preferred compositions of the present invention incorporate a pharmaceutically effective amount of nicotine levulinate. For a composition of the present invention incorporating nicotine levulinate, the amount of nicotine attributable to the nicotine levulinate typically is at least about 10 percent, and often at least about 20 percent of the total amount of nicotine active ingredient within the composition (calculated as nicotine base), on a weight basis. For a composition incorporating nicotine levulinate, the amount of nicotine attributable to the nicotine levulinate typically does not exceed about 75 percent, and often does not exceed about 50 percent of the total amount of nicotine active ingredient (calculated as nicotine base) within the composition.

The dose of active ingredient (i.e., all the various nicotine forms) is that amount effective to treat some symptoms of, or prevent occurrence of the symptoms of, the condition, disease, or disorder from which the subject or patient suffers. By “effective amount”, “therapeutic amount” or “effective dose” is meant that amount sufficient to elicit the desired pharmacological or therapeutic effects, thus resulting in effective prevention or treatment of the condition, disease, or disorder. Thus, an effective amount of active ingredient is an amount sufficient to enter relevant regions of the body (e.g., including passing across the blood-brain barrier of the subject), to bind to relevant receptor sites in the CNS and PNS of the subject, and/or to elicit neuropharmacological effects (e.g., elicit neurotransmitter secretion, thus resulting in effective prevention or treatment of the condition, disease, or disorder). Prevention of the disorder is manifested, for example, by delaying the onset of the symptoms of the condition, disease, or disorder. Treatment of the disorder is manifested by, for example, a decrease in the symptoms associated with the condition, disease, or disorder or an amelioration of the reoccurrence of the symptoms thereof.

For compositions of the present invention, the intended daily dose of the active ingredient can vary. The overall dose of active ingredient can depend upon factors such as the weight of the subject ingesting the composition, the type of condition, disease, or disorder being treated, the state or severity of the condition, disease, or disorder being treated, the desired pharmacological effect, or other such factors. Typically, the amount of nicotine active ingredient, calculated as nicotine base, administered to a subject per day is at least about 2 mg, often is at least about 4 mg, and frequently is at least about 10 mg. Typically, the amount of nicotine active ingredient administered to a subject per day does not exceed about 60 mg, often does not exceed about 50 mg, and frequently does not exceed about 40 mg. See also, for example, the types of dosing regimens and administration techniques set forth in U.S. Pat. No. 5,593,684 to Baker et al. and U.S. Pat. No. 6,660,754 to Kyle et al.; and US Pat. Pub. Nos. 2004/0006113 to Sachs; 2005/0214229 to Pinney et al.; 2008/0124283 to Andersen and 2009/0293895 to Axelsson et al.; which are incorporated herein by reference.

In use, the compositions of the present invention are typically administered in a form adapted for buccal, sublingual, or nasal delivery. In certain embodiments, the compositions are in a form suitable for oral ingestion. For example, nicotine-containing compositions can be administered and employed using the manners and methods typically used for the administration of traditional types of nicotine-containing gums, lozenges, pouch products, and sprays. As previously noted, the incorporation of a levulinate moiety into nicotine-containing compositions intended to be administered by oral means can provide for amelioration of the types of dissonant sensory and organoleptic effects attributed to the administration of nicotine. Thus, a sufficient pharmaceutically acceptable dosage of nicotine can be administered orally due to the improved palatability attributed to the presence of an effective amount of levulinate moiety. However, it is preferred that the amount of levulinate moiety is not overly great so as to ensure that the characteristic organoleptic and sensory attributes of nicotine are experienced by the human subject orally ingesting the composition. As such, it is highly preferred that there exists a nicotine source in addition to nicotine levulinate, and that the levulinate moiety excipient (and other excipients) not be present at a concentration so high that none of the organoleptic and sensory attributes of the nicotine are detectable in the final composition.

The compositions of the present invention can be used for treatment of a wide variety of conditions, diseases, and disorders responsive to stimulation of one or more types of nicotinic acetylcholinergic receptors (nAChRs). The compositions can be used to treat those types of conditions, diseases, and disorders that have been reported to be treatable through the use or administration of nicotine as an agonist of nAChRs. As such, the compositions can be used to treat various CNS conditions, diseases, and disorders, and the compositions also can be used as smoking cessation aids (i.e., as components of NRT).

### EXAMPLES

The following examples are provided in order to further illustrate the invention but should not be construed as limiting the scope thereof. Unless otherwise noted, all parts and percentages are by weight. For each example employing nicotine levulinate as a component, the nicotine levulinate is produced using the types of materials and techniques set forth in Example 1 of U.S. Pat. No. 4,830,028 to Lawson et al. (i.e., the nicotine is virtually all in the form of 1-nicotine). The following examples are illustrative of representative products that can be employed to provide for oral ingestion of nicotine for therapeutic purposes, such as NRT.

### Example 1

A gum generally similar in shape and form to a nicotine-containing gum incorporating 4 mg of nicotine and commercially available as Nicorette Original Gum (distributed by GlaxoSmithKline Consumer Healthcare, L.P.) is produced using generally similar excipient ingredients and processing conditions used for the manufacture of the commercial gum, except that the nicotine polacrilex thereof is replaced by a mixture of nicotine polacrilex and nicotine levulinate. The amount of nicotine polacrilex incorporated into each chewing piece of gum is such that the amount of nicotine active ingredient within each chewing piece from that source is 3 mg; and the amount of nicotine levulinate incorporated into each chewing piece of gum is such that the amount of nicotine active ingredient within each chewing piece from that source is 1 mg. As such, each chewing piece of the gum product incorporates 4 mg of nicotine active ingredient. Each unit or chewing piece incorporates forms of nicotine from two sources.

### Example 2

A coated gum generally similar in shape and form to a nicotine-containing gum incorporating 4 mg of nicotine and commercially available as Coated Nicotine Gum (distributed by Walgreen Co.) is produced using generally similar excipient ingredients and processing conditions used for the manufacture of the commercial gum, except that the nicotine polacrilex thereof is replaced by a mixture of nicotine polacrilex and nicotine levulinate. The amount of nicotine polacrilex incorporated into each chewing piece of gum is such that the amount of nicotine active ingredient within each chewing piece from that source is 3 mg; and the amount of nicotine levulinate incorporated into each chewing piece of gum is such that the amount of nicotine active ingredient within each chewing piece from that source is 3 mg. As such, each chewing piece of the coated gum product incorporates 6 mg of nicotine active ingredient.

### Example 3

A coated gum generally similar in shape and form to a nicotine-containing gum incorporating 4 mg of nicotine and commercially available as Nicorette Fruit Chill Gum (distributed by Walgreen Co.) is produced using generally similar excipient ingredients and processing conditions used for the manufacture of the commercial gum, except that the nicotine polacrilex thereof is replaced by a mixture of nicotine polacrilex and nicotine levulinate. The amount of nicotine polacrilex incorporated into each chewing piece of gum is such that the amount of nicotine active ingredient within each chewing piece from that source is 3 mg; and the amount of nicotine levulinate incorporated into each chewing piece of gum is such that the amount of nicotine active ingredient within each chewing piece from that source is 2 mg. As such, each chewing piece of the coated gum product incorporates 5 mg of nicotine active ingredient.

### Example 4

A coated gum generally similar in shape and form to a nicotine-containing gum incorporating 4 mg of nicotine and commercially available as Nicorette Fruit Chill Gum (distributed by Walgreen Co.) is produced using generally similar excipient ingredients and processing conditions used for the manufacture of the commercial gum, except that the nicotine polacrilex thereof is replaced by a mixture of nicotine polacrilex and nicotine levulinate. In addition, 0.5 mg of levulinate moiety in the form of sodium levulinate is incorporated into the composition. The amount of nicotine polacrilex incorporated into each chewing piece of gum is such that the amount of nicotine active ingredient within each chewing piece from that source is 3 mg; and the amount of nicotine levulinate incorporated into each chewing piece of gum is such that the amount of nicotine active ingredient with each chewing piece from that source is 2 mg. As such, each chewing piece of the coated gum product incorporates 5 mg of nicotine active ingredient. Each unit or chewing piece incorporates forms of levulinate moiety in two forms from two sources.

### Example 5

A coated gum generally similar in shape and form to a nicotine-containing gum incorporating 4 mg of nicotine and commercially available as Zonnic (distributed by Niconovum AB) is produced using generally similar excipient ingredients and processing conditions used for the manufacture of the commercial gum, except that the nicotine and microcrystalline cellulose thereof is replaced by a mixture of nicotine/microcrystalline cellulose and nicotine levulinate. The amount of nicotine/microcrystalline cellulose incorporated into each chewing piece of gum is such that the amount of nicotine active ingredient within each chewing piece from that source is 3 mg; and the amount of nicotine levulinate incorporated into each chewing piece of gum is such that the amount of nicotine active ingredient within each chewing piece from that source is 2 mg. As such, each chewing piece of the coated gum product incorporates 5 mg of nicotine active ingredient.

### Example 6

A gum generally similar in shape and form to a nicotine-containing gum incorporating 4 mg of nicotine and commercially available as Nicorette Original Gum (distributed by GlaxoSmithKline Consumer Healthcare, L.P.) is produced using generally similar excipient ingredients and processing conditions used for the manufacture of the commercial gum, except that 0.5 mg of sodium levulinate is incorporated within the formulation employed to manufacture that gum. As such, an excipient possessing a levulinate moiety is incorporated within the overall composition of that gum product.

### Example 7

A gum product generally similar in shape and form, and produced using generally similar excipient ingredients and processing conditions, to the nicotine-containing gum designated as Comp. A as set forth in Example 6 of US Pat. Pub. No. 2010/0061940 to Axelsson et al. is provided, except that, in addition to the nicotine ingredient of that lozenge, sufficient nicotine levulinate is incorporated into each lozenge such that the total amount of nicotine active ingredient with each gum chewing piece or unit is 4 mg.

### Example 8

A gum product generally similar in shape and form, and produced using generally similar excipient ingredients and processing conditions, to the nicotine-containing gum designated as Comp. B, as set forth in Example 6 of US Pat. Pub. No. 2010/0061940 to Axelsson et al. is provided, except that, in addition to the nicotine ingredient of that lozenge, sufficient nicotine levulinate is incorporated into each lozenge such that the total amount of nicotine active ingredient with each gum chewing piece or unit is 2.5 mg.

### Example 9

A lozenge generally similar in shape and form to a nicotine-containing lozenge incorporating 2 mg of nicotine and commercially available as Nicotine polacrilex Lozenge (distributed by CVS Pharmacy, Inc.) is produced using generally similar excipient ingredients and processing conditions used for the manufacture of the commercial lozenge, except that the nicotine polacrilex active ingredient replaced by a mixture of nicotine polacrilex and nicotine levulinate. The amount of nicotine polacrilex incorporated into each lozenge is such that the amount of nicotine active ingredient within each lozenge from that source is 2 mg; and the amount of nicotine levulinate incorporated into each lozenge is such that the amount of nicotine active ingredient within each lozenge from that source is 2 mg. As such, the lozenge product incorporates 4 mg of nicotine active ingredient, per lozenge.

### Example 10

A lozenge generally similar in shape and form to a nicotine-containing lozenge incorporating 2 mg of nicotine and commercially available as Nicotine polacrilex Lozenge (distributed by CVS Pharmacy, Inc.) is produced using generally similar excipient ingredients and processing conditions used for the manufacture of the commercial lozenge, except that the nicotine polacrilex active ingredient replaced by a mixture of nicotine polacrilex and nicotine levulinate. The amount of nicotine polacrilex incorporated into each lozenge is such that the amount of nicotine active ingredient within each lozenge from that source is 2 mg; and the amount of nicotine levulinate incorporated into each lozenge is such that the amount of nicotine active ingredient within each lozenge from that source is 3 mg. As such, the lozenge product incorporates 5 mg of nicotine active ingredient, per lozenge.

### Example 11

A lozenge generally similar in shape and form to a nicotine-containing lozenge incorporating 2 mg of nicotine and commercially available as Nicotine polacrilex Lozenge (distributed by CVS Pharmacy, Inc.) is produced using generally similar excipient ingredients and processing conditions used for the manufacture of the commercial lozenge, except that the nicotine polacrilex active ingredient replaced by a mixture of nicotine polacrilex and nicotine levulinate. The amount of nicotine polacrilex incorporated into each lozenge is such that the amount of nicotine active ingredient within each lozenge from that source is 2 mg; and the amount of nicotine levulinate incorporated into each lozenge is such that the amount of nicotine active ingredient within each lozenge from that source is 3 mg. In addition, 0.5 mg of levulinate moiety in the form of sodium levulinate is incorporated into the composition. As such, the lozenge product incorporates 5 mg of nicotine active ingredient, per lozenge.

### Example 12

A lozenge generally similar in shape and form to a nicotine-containing lozenge incorporating 2.5 mg of nicotine is produced using generally similar excipient ingredients and processing conditions used for the manufacture of that lozenge set forth in Table 1 of Example 3 of US Pat. Pub. No. 2010/0004294 to Axelsson et al., except that, in addition to the nicotine bitartrate dihydrate ingredient of that lozenge, sufficient nicotine levulinate is incorporated into each lozenge such that the total amount of nicotine active ingredient within each lozenge is 3.5 mg.

### Example 13

A lozenge generally similar in shape and form to a nicotine-containing lozenge incorporating 2.5 mg of nicotine is produced using generally similar excipient ingredients and processing conditions used for the manufacture of that lozenge set forth in Table 1 of Example 3 of US Pat. Pub. No. 2010/0004294 to Axelsson et al., except that 1 mg of sodium levulinate is incorporated within the formulation employed to manufacture that lozenge. As such, an excipient possessing a levulinate moiety is incorporated within the overall composition of that lozenge product.

### Example 14

A lozenge generally similar in shape and form to a nicotine-containing lozenge incorporating 2 mg of nicotine and commercially available as NiQuitin (distributed by GSK Consumer Healthcare A/S) is produced using generally similar excipient ingredients and processing conditions used for the manufacture of the commercial lozenge, except that the nicotine bitartrate active ingredient replaced by a mixture of nicotine bitartrate and nicotine levulinate. The amount of nicotine bitartrate incorporated into each lozenge is such that the amount of nicotine active ingredient within each lozenge from that source is 2 mg; and the amount of nicotine levulinate incorporated into each lozenge is such that the amount of nicotine active ingredient with each lozenge from that source is 1 mg. As such, the lozenge product incorporates 3 mg of nicotine active ingredient, per lozenge.

### Example 15

A pouch type of product similar in shape and form to a nicotine-containing pouch commercially available as Zonnic (distributed by Niconovum A.B.) is produced using generally similar pouch material, excipient ingredients and processing conditions used for the manufacture of the commercial pouch, except that the nicotine/microcrystalline cellulose ingredient thereof is replaced by a mixture of nicotine levulinate and nicotine/microcrystalline cellulose. The amount of nicotine/microcrystalline cellulose incorporated into each pouch is such that the amount of nicotine active ingredient within each pouch from that source is the same as the commercially available pouch, and the amount of nicotine levulinate incorporated into the pouch is such that the overall nicotine content of product is doubled (as compared to the commercially available product). In addition, 0.5 mg of levulinate moiety in the form of sodium levulinate is incorporated into the composition.

### Example 16

A pouch type of product similar in shape and form to a nicotine-containing pouch commercially available as Zonnic (distributed by Niconovum A.B.) is produced using generally similar pouch material, excipient ingredients and processing conditions used for the manufacture of the commercial pouch, except that the nicotine/microcrystalline cellulose ingredient thereof is replaced by a mixture of nicotine levulinate and nicotine/microcrystalline cellulose. The amount of nicotine/microcrystalline cellulose incorporated into each pouch is such that the amount of nicotine active ingredient within each pouch from that source is the same as the commercially available pouch, and the amount of nicotine levulinate incorporated into the pouch is such that the overall nicotine content of product is doubled (as compared to the commercially available product).

### Example 17

Pouch type products generally similar in shape and form to a nicotine-containing pouches set forth as snuff bag compositions E-J in Example 1 of PCT WO 2007/104573 to Axelsson et al. are produced using generally similar excipient ingredients and processing conditions used for the manufacture of those pouch type products, except that 2 mg of sodium levulinate is incorporated within the formulation employed to manufacture that pouch product. As such, an excipient possessing a levulinate moiety is incorporated within the overall composition of that pouch product.

### Example 18

Pouch type products generally similar in shape and form to nicotine-containing pouches set forth as snuff bag compositions E-J in Example 1 of US Pat. Pub. No. 2009/0293895 to Axelsson et al. are produced using generally similar excipient ingredients and processing conditions used for the manufacture of those pouch type products, except that an additional 1 mg of nicotine is incorporated within the formulation employed to manufacture that pouch product. The additional nicotine is provided by the addition of a sufficient amount of nicotine levulinate to provide the 1 mg of nicotine. Each pouch type product possesses about 7 mg nicotine. As such, an excipient possessing a levulinate moiety is incorporated within the overall composition of that pouch product.

### Example 19

A spray formulation generally similar to a nicotine-containing spray formulation designated as Composition A and set forth in Example 1 of US Pat. Pub. No. 2009/0023819 to Axelsson is prepared, except that 0.5 mg of sodium levulinate is incorporated into that formulation.

### Example 20

A spray formulation generally similar to a nicotine-containing spray formulation commercially available as Zonnic (distributed by Niconovum A.B.) is prepared, except that an additional 0.5 mg of nicotine and 1 mg of sodium levulinate is incorporated into that formulation.

### Example 21

A spray formulation generally similar to a nicotine-containing spray formulation commercially available as Zonnic (distributed by Niconovum A.B.) is prepared, except that sufficient nicotine levulinate is incorporated into the formulation to double the amount of nicotine within the formulation.

What is claimed is:

1. A nicotine-containing pharmaceutical composition, comprising:
a source of nicotine and
a levulinate moiety,
wherein the composition is in a pharmaceutically acceptable form adapted for oral or nasal delivery of the composition.

2. The pharmaceutical composition of claim 1, wherein the levulinate moiety has the form of levulinic acid.

3. The pharmaceutical composition of claim 1, wherein the levulinate moiety has the form of a levulinate salt.

4. The pharmaceutical composition of claim 3, wherein the levulinate salt is an alkali metal or alkali earth metal salt.

5. The pharmaceutical composition of claim 1, wherein the levulinate moiety has the form of an ester of levulinic acid.

6. The pharmaceutical composition of claim 1, wherein the levulinate moiety is incorporated within nicotine levulinate.

7. The pharmaceutical composition of claim 1, wherein the source of nicotine is nicotine polacrilex and the levulinate moiety is incorporated within nicotine levulinate.

8. The pharmaceutical composition of claim 1, wherein the source of nicotine is in a free base form and the levulinate moiety is incorporated within nicotine levulinate.

9. The pharmaceutical composition of claim 1, wherein the source of nicotine is a nicotine salt and the levulinate moiety is nicotine levulinate.

10. The pharmaceutical composition of claim 9, wherein the source of nicotine is nicotine tartrate or nicotine bitartrate.

11. The pharmaceutical composition of claim 1, wherein the source of nicotine is in the form of a free base, a salt, a complex, or a solvate.

12. The pharmaceutical composition of claim 1, wherein one or both of the source of nicotine and the levulinate moiety are sorbed onto a porous particulate carrier.

13. The pharmaceutical composition of claim 12, wherein the porous particulate carrier comprises microcrystalline cellulose.

14. The pharmaceutical composition of claim 12, wherein nicotine free base and nicotine levulinate are sorbed onto the porous particulate carrier.

15. The pharmaceutical composition of claim 1, wherein the composition is in a form adapted for oral ingestion.

16. The pharmaceutical composition of claim 15, wherein the composition is in the form of a gum.

17. The pharmaceutical composition of claim 15, wherein the composition is in the form of a lozenge.

18. The pharmaceutical composition of claim 15, wherein the composition is in the form of a tablet.

19. The pharmaceutical composition of claim 15, wherein the composition is in the form of a spray.

20. The pharmaceutical composition of claim 15, wherein the composition is in the form of a pouch product.

21. A nicotine-containing pharmaceutical composition, comprising:
a source of nicotine selected from the group consisting of nicotine in free base form, a nicotine salt other than nicotine levulinate, a resin complex of nicotine, and mixtures thereof, and
a levulinate moiety selected from the group consisting of levulinic acid, nicotine levulinate, an alkali metal or alkali earth metal salt of levulinic acid, an alkyl ester of levulinic acid, and mixtures thereof;
wherein the composition is in a pharmaceutically acceptable form adapted for oral ingestion of the composition.

22. A method for treating a human subject having a condition, disease, or disorder responsive to stimulation of nicotinic acetylcholinergic receptors, comprising orally or nasally administering an effective amount of a pharmaceutical composition according to claim 1 to said human subject.

23. The method of claim 22, wherein said administering step comprises administering the pharmaceutical composition to a human subject as a smoking cessation aid.

24. The method of claim 22, wherein the levulinate moiety has the form of levulinic acid.

25. The method of claim 22, wherein the levulinate moiety has the form of a levulinate salt.

26. The method of claim 22, wherein the levulinate moiety has the form of an ester of levulinic acid.

27. The method of claim 22, wherein the levulinate moiety is incorporated within nicotine levulinate.

28. The method of claim 22, wherein the source of nicotine is nicotine polacrilex and the levulinate moiety is incorporated within nicotine levulinate.

29. The method of claim 22, wherein the source of nicotine is in a free base form and the levulinate moiety is incorporated within nicotine levulinate.

30. The method of claim 22, wherein the source of nicotine is a nicotine salt and the levulinate moiety is nicotine levulinate.

31. The method of claim 30, wherein the source of nicotine is nicotine tartrate or nicotine bitartrate.

32. The method of claim 22, wherein one or both of the source of nicotine and the levulinate moiety are sorbed onto a porous particulate carrier.

33. The method of claim 32, wherein the porous particulate carrier comprises microcrystalline cellulose.

34. The method of claim 32, wherein nicotine free base and nicotine levulinate are sorbed onto the porous particulate carrier.

35. The method of claim 22, wherein the composition is in a form adapted for oral ingestion.

36. The method of claim 35, wherein the composition is in the form of a gum.

37. The method of claim 35, wherein the composition is in the form of a lozenge.

38. The method of claim 35, wherein the composition is in the form of a tablet.

39. The method of claim 35, wherein the composition is in the form of a spray.

40. The method of claim 35, wherein the composition is in the form of a pouch product.
